(19) United States
(12) Patent Application Publication
(10) Pub. No.: US 2012/0150117 A1
(43) Pub. Date: Jun. 14, 2012

(54) INFUSION LINE

(75) Inventor: Rafael V. Andino, Grayson, GA (US)

(73) Assignee: Venetec International, Inc., Covington, GA (US)

(21) Appl. No.: 13/315,029

(22) Filed: Dec. 8, 2011

Related U.S. Application Data
(60) Provisional application No. 61/423,013, filed on Dec. 14, 2010.

(51) Int. Cl. A61B 17/34

(52) U.S. Cl. 604/164.01

(57) ABSTRACT

An intravenous (IV) line ratchet system is used to start an intravenous line to introduce or withdraw fluids from the vasculature of a patient. The IV line ratchet system includes a rotating member that causes a needle and catheter to be inserted into the vasculature of the patient. The rotating member further causes the needle to be removed from the vasculature of the patient while the catheter remains in the vasculature of the patient.

### RELATED APPLICATIONS

This application claims priority under 35 U.S.C. §119(e) to U.S. Provisional Patent Application Ser. No. 61/423,013, filed Dec. 14, 2010, and entitled INFUSION LINE, which is hereby expressly incorporated by reference in its entirety.

### BACKGROUND

1. Field of the Invention

This invention relates to a drug infusion line. More specifically, this invention relates to a ratchet system for starting an intravenous line in order to deliver therapeutic agents to a patient.

2. Description of the Related Art

It is common for medical professionals to start intravenous (“I.V.”) lines to introduce fluids and medications directly into a patient or to withdraw fluids from the patient. Depending on where they are employed, healthcare professionals may be tasked with starting dozens of I.V. lines on any given day. Different I.V. lines require different combinations of equipment and it can be difficult and time consuming for a healthcare provider to gather different combinations of equipment throughout the day. The time spent gathering equipment used to start an I.V. line can be stressful for both patients and healthcare providers. For example, in emergency situations, any time used to gather I.V. line start equipment can pose serious risks for the patient.

Further, the procedure of starting an I.V. line can be difficult and time consuming. The healthcare professional must be properly trained to identify where to insert the I.V. line as well as how to insert the line. Improper insertion can lead to problems of fluids being introduced into the tissue instead of the vasculature of the patient. Further, improper insertion may require the I.V. line be removed and inserted again, which may cause discomfort to the patient.

For these reasons, a need exists for an improved way to insert IV lines into patients.

### SUMMARY

The devices and methods of the present invention have several features, no single one of which is solely responsible for its desirable attributes. Without limiting the scope of this invention as expressed by the claims which follow, its more prominent features will now be discussed briefly. After considering this discussion, and particularly after reading the section entitled “Detailed Description of Certain Embodiments,” one will understand how the features of this invention provide several advantages over existing medical devices.

An aspect of the invention includes an intravenous line starting system that has a housing, a first rotating member disposed in the housing, and a catheter connected to the first rotating member. The system further includes a second rotating member disposed in the housing, a needle connected to the second rotating member, and an actuator configured to rotate the first rotating member and the second rotating member between at least a first position, a second position, and a third position. When in the first position the catheter and the needle are generally located in the housing. When in the second position the catheter and the needle extend beyond the housing. When in the third position the catheter extends beyond the housing and the needle is generally located in the housing.

An aspect of the invention includes an intravenous line starting system that has a housing including a plurality of members configured to rotate about a common axis and a catheter engaged with one of the plurality of rotating members. The system further includes a needle disposed within the catheter and engaged with another one of the plurality of members. Rotation of at least one of the plurality of members about the common axis extends and retracts the needle relative to the housing without rotating the needle.

An aspect of the invention includes an intravenous line starting device that has a first member engaged with a needle and a second member engaged with a catheter. The needle is coaxially disposed within the catheter. Rotation of the first member extends and retracts the needle and rotation of the second member extends and retracts the catheter.

### BRIEF DESCRIPTION OF THE DRAWINGS

The above mentioned and other features of the invention will now be described with reference to the drawings of several embodiments of the present IV ratchet system. The illustrated embodiments of the infusion system are intended to illustrate, but not to limit the invention. The drawings contain the following figures:

FIG. 1 is a side view of one embodiment of an IV ratchet system in accordance with the disclosure herein.

FIG. 2 is another side view of the IV ratchet system of FIG. 1.

FIG. 3 is another side view of the IV ratchet system of FIG. 1.

FIG. 4 is bottom view of the IV ratchet system of FIG. 1.

FIG. 5 is a cross sectional view of the IV ratchet system of FIG. 1.

### DETAILED DESCRIPTION OF CERTAIN EMBODIMENTS

The following description and the accompanying figures, which describe and show the preferred embodiments, are made to demonstrate several possible configurations that an I.V. ratchet system can take to include various aspects and features of the invention. Those of skill in the art will recognize that the disclosed aspects and features of the invention are not limited to any particular embodiment of an I.V. ratchet system, which can include one or more of the inventive aspects and features herein described, and can be designed for a variety of medical procedures.

The preferred embodiments of the present invention advantageously provide an IV ratchet system for starting an IV line into a patient and methods for using an IV ratchet system. The IV ratchet system includes a needle and a catheter. The IV ratchet system includes a housing with a movable lever that controls movement of the needle and catheter. Movement of the lever causes the needle and catheter to enter the vasculature of a patient and further retracts the needle from the vasculature. Accordingly, the catheter is inserted into the vasculature of the patient. The catheter is further connected to a medical line to allow fluids to be withdrawn or inserted to the vasculature via the medical line.

To facilitate a complete understanding of the preferred embodiments, the remainder of the detailed description describes the IV ratchet system and methods of using the same with reference to the figures, wherein like elements among embodiments are referenced with like numerals throughout the following description

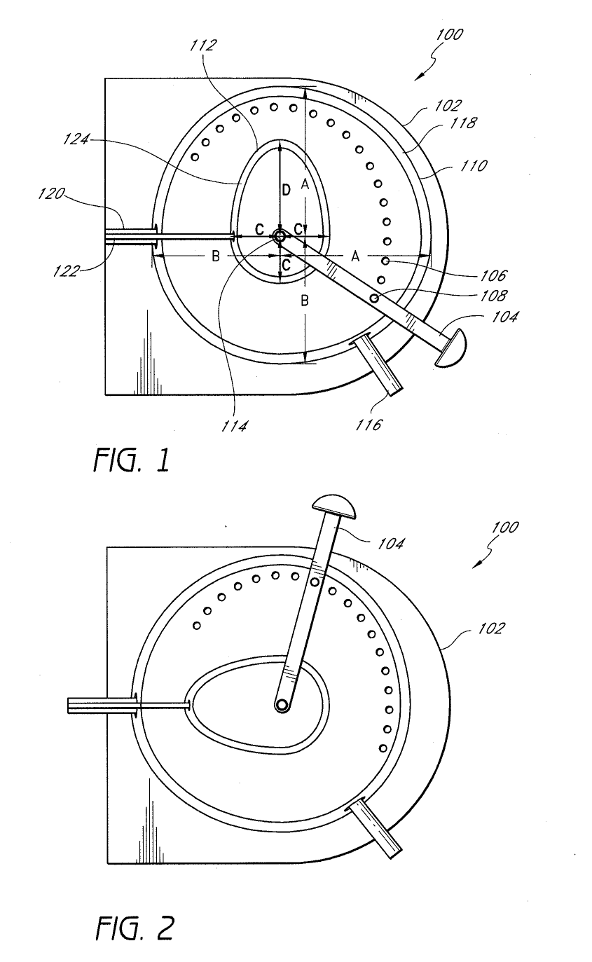

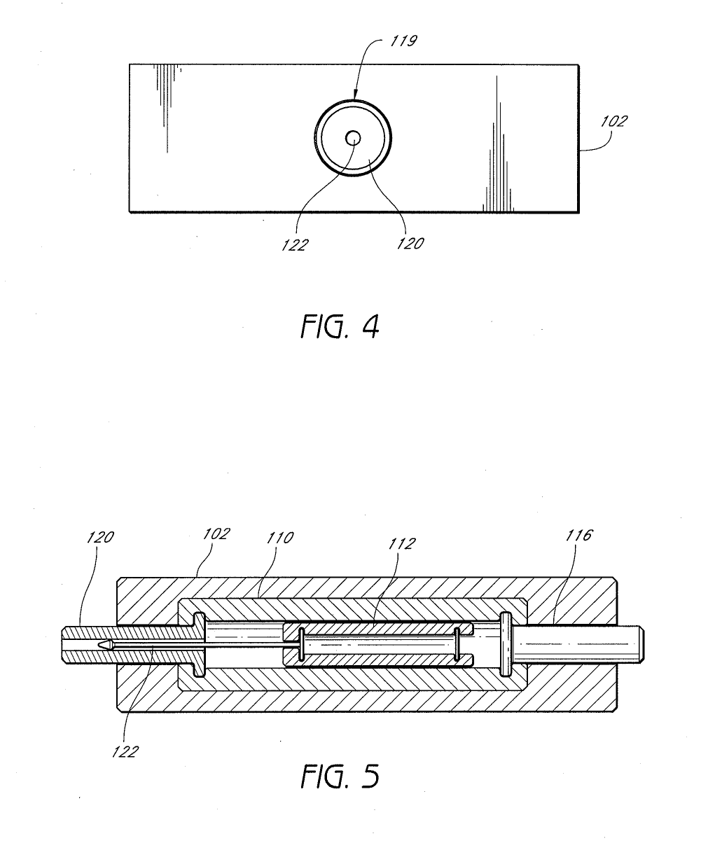

FIGS. 1-5 show an IV ratchet system 100 which includes a housing 102. In one embodiment the housing 102 is made of plastic. The housing 102 may further be coated with an antimicrobial coating. The IV ratchet system 100 further includes a lever 104 affixed to the housing 102. The lever 104 is rotatable with respect to the housing 102. The housing 102 further includes a series of engaging members 106, or impressions, that are configured to mate with an engaging member 108 located on the lever 104. The engaging members 106 and 108 may comprise different structures that interlock with each other to inhibit free movement of the lever 104 with respect to the housing 102 such as grooves or notches and corresponding protrusions. Based on the interaction between the engaging members 106 and 108, the lever 104 may be held in a variety of positions as it is rotated with respect to the housing 102.

The lever 104 is further connected to rotating members 110 and 112. Rotating members 110 and 112 may be connected to the lever 104 by means of a common axis 114 formed at the center of the housing 102 that extends in the transverse direction. Movement of the lever 104 may cause each of the rotating members 110 and 112 to rotate within the housing 102 about the axis 114.

The rotating member 110 may be connected to a medical line 116. The housing 102 includes an opening for the medical line 116 to connect to the rotating member 110 and pass out of the housing 102. The connection between the rotating member 110 and the medical line 116 is configured to allow the rotating member 110 to rotate with respect to the medical line 116, without causing the medical line 116 to rotate. In one embodiment, the medical line 116 may connect to a channel 118 formed in the outer portion of the rotating member 110. At least a portion of the medical line 116 may be configured to move along the channel 118 as the rotating member 110 rotates. Accordingly, the medical line 116 remains in the same position with respect to the housing 102 as the rotating member 110 rotates.

The rotating member 110 may further be connected to a catheter. The housing 102 includes an opening 119 for the catheter 120 to connect to the rotating member 110 and pass through the housing 102. The connection between the rotating member 110 and the catheter 120 is configured to allow the rotating member 110 to rotate with respect to the catheter 120, without causing the catheter 120 to rotate. In one embodiment, the catheter 120 may connect to the channel 118. At least a portion of the catheter 120 may be configured to move along the channel 118 as the rotating member 110 rotates. Accordingly, the catheter 120 remains in the same position with respect to the housing 102 as the rotating member 110 rotates. The catheter 120, however, may move in a longitudinal direction as the rotating member 110 rotates. In particular as the rotating member 110 is rotated such that a portion of the rotating member 110 with a greater radius is aligned with the longitudinal axis of the catheter 120, the catheter 120 moves in a longitudinal direction outward from the housing 102. As the rotating member 110 is rotated such that a portion of the rotating member 110 with a lesser radius is aligned with the longitudinal axis of the catheter 120, the catheter 120 moves in a longitudinal direction inward from the housing 102. The rotating member 110 may be configured to have at least two portions with a radius A and at least one portion with a radius B, wherein A is greater than B.

The rotating member 112 may further be in connection with a needle 122. The needle 122 may be configured to fit inside the catheter 120. Further, the rotating member 112 and needle 122 may fit inside the rotating member 110. The needle 122 may pass through the same opening 119 of the housing 102 as the catheter 120. The connection between the rotating member 112 and the needle 122 is configured to allow the rotating member 112 to rotate with respect to the needle 122, without causing the needle 122 to rotate. In one embodiment, the needle 122 may connect to a channel 124 formed in the outer portion of the rotating member 112. At least a portion of the needle 122 may be configured to move along the channel 124 as the rotating member 112 rotates. Accordingly, the needle 122 remains in the same position with respect to the housing 102 as the rotating member 112 rotates. The needle 122, however, may move in a longitudinal direction as the rotating member 112 rotates. In particular as the rotating member 112 is rotated such that a portion of the rotating member 112 with a greater radius is aligned with the longitudinal axis of the needle 122, the needle 122 moves in a longitudinal direction outward from the housing 102. As the rotating member 112 is rotated such that a portion of the rotating member 110 with a lesser radius is aligned with the longitudinal axis of the needle 122, the needle 122 moves in a longitudinal direction inward from the housing 102. The rotating member 112 may be configured to have at least two portions with a radius C and at least one portion with a radius D, wherein D is greater than C. Further, the at least one portion of the rotating member 112 with the radius D may be aligned with one portion of the rotating member 110 with the radius A. Further, one portion of the rotating member 112 with the radius C may be aligned with one portion of the rotating member with the radius B, and the other portion of the rotating member 112 with the radius C may be aligned with one portion of the rotating member with the radius A.

The IV ratchet system 100 may be used as discussed below to start an IV in a patient and withdraw or introduce fluids to the vasculature of the patient.

The IV ratchet system 100 may be placed on the skin of a patient, such as on the arm, over an area of tissue that includes some type of vasculature. The housing 102 may be placed on the skin with the opening 119 placed over the vasculature. During initial placement of the housing 102 on the skin, the lever 104 may be rotated such that the needle 122 and the catheter 120 are both positioned within the housing 102 as shown in FIG. 1. As discussed above, the lever 104 may be moved causing the rotating members 110 and 112 to rotate. Such rotation of the rotating member 110 and 112 causes the catheter 120 and the needle 122 to move in a longitudinal direction.

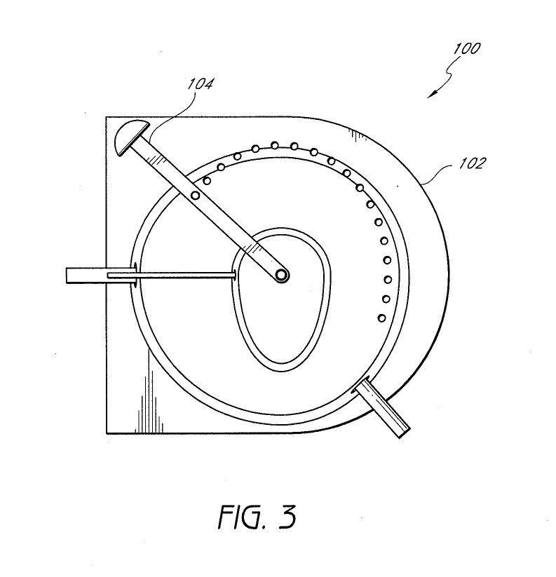

Continuing, the lever 104 may be rotated such that the needle 122 and the catheter 120 move together in the longitudinal direction through the skin and tissue of the patient, and into the vasculature of the patient as shown in FIG. 2. As discussed above, the rotating members 110 and 112 are specifically shaped to effect such movement. The lever 104 may be further rotated such that the needle 122 moves back into the housing 102 and the catheter 120 remains in the vasculature of the patient as shown in FIG. 3. The medical line 116, rotating member 110, and the catheter 120 may form a fluid channel that allows fluids to be passed between the medical line 116 and the catheter 120. Accordingly, fluid may pass between the vasculature and the medical line 116.

Of course, it is to be understood that not necessarily all objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Furthermore, the skilled artisan will recognize the interchangeability of various features from different embodiments. In addition to the variations described herein, other known equivalents for each feature can be mixed and matched by one of ordinary skill in this art to construct stabilization systems and techniques in accordance with principles of the present invention.

Although this invention has been disclosed in the context of certain embodiments and examples, it will be understood by those skilled in the art that the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above, but by a fair reading of the claims that follow.

What is claimed is:

1. A intravenous line starting system comprising:
a housing;
a first rotating member disposed in the housing;
a catheter connected to the first rotating member;
a second rotating member disposed in the housing;
a needle connected to the second rotating member; and
an actuator configured to rotate the first rotating member and the second rotating member between at least a first position, a second position, and a third position, when in the first position the catheter and the needle are generally located in the housing, when in the second position the catheter and the needle extend beyond the housing, and when in the third position the catheter extends beyond the housing and the needle is generally located in the housing.

2. The system of claim 1, wherein the actuator comprises an engagement member configured to engage with a portion of the housing in order to inhibit movement of the actuator.

3. The system of claim 1, wherein rotation of the second rotating member causes longitudinal movement of the needle.

4. The system of claim 1, wherein rotation of the first rotating member causes longitudinal movement of the catheter.

5. The system of claim 1, wherein at least a portion of the second rotating member is disposed in the first rotating member.

6. The system of claim 1, wherein the catheter, medical line, and the first rotating member form a lumen.

7. The system of claim 6, wherein the lumen is configured to pass fluid.

8. The system of claim 1, wherein the first rotating member and the second rotating member rotate about a common axis.

9. The system of claim 1 further comprising a medical line in flow communication with the catheter.

10. A intravenous line starting system comprising:
a housing including a plurality of members configured to rotate about a common axis;
a catheter engaged with one of the plurality of rotating members; and
a needle disposed within the catheter and engaged with another one of the plurality of members, wherein rotation of at least one of the plurality of members about the common axis extends and retracts the needle relative to the housing without rotating the needle.

11. The system of claim 10 further comprising an actuator, the actuator being configured to rotate at least one of the plurality of rotating members.

12. The system of claim 10, wherein the needle extends and retracts along a longitudinal axis of the needle.

13. The system of claim 10, wherein rotation of the at least one of the plurality of members extends and retracts the catheter relative to the housing.

14. The system of claim 10, wherein at least one of the plurality of rotating members is disposed within another one of the plurality of rotating members.

15. The system of claim 10 further comprising an outlet configured to connect to a medical line, the outlet being in flow communication with the catheter.

16. The system of claim 10, wherein at least one of the plurality of rotating members comprises a channel, the needle being configured to slide through the channel when the at least one of the plurality of rotating members is rotated.

17. A intravenous line starting device comprising a first member engaged with a needle and a second member engaged with a catheter, the needle being coaxially disposed within the catheter, wherein rotation of the first member extends and retracts the needle, and wherein rotation of the second member extends and retracts the catheter.

18. The device of claim 17, wherein during at least a portion of the rotation of the first member the needle moves independent of the catheter.

19. The device of claim 17 further comprising a medical line, the medical line being in fluid communication with the catheter.

20. The device of claim 17, wherein the first member and the second member rotate about a common axis.
